SEQUENCE LISTING (12 sequences)

SEQ ID NO: 1 - protein, 264 aa, Artificial Sequence
  Description of Artificial Sequence: Synthetic polypeptide
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGAGGGGAGGGGSEVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 2 - DNA, 792 bp, Artificial Sequence
  Description of Artificial Sequence: Synthetic polynucleotide
gaggtgcagctggttgaatctggcggaggacttgtgaagcctggcggctctctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaagggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttacagtttctagtggcggaggcggagctggtggtggcggagcaggaggcggaggatctgaagttcaacttgtcgagagtggcggcggactggttcaaccaggcggaagtcttagactgagctgtgctgctagcggcagagcccactctgattacgctatggcatggttcaggcaggccccaggacaagaaagggaatttgtggccggaatcggttggagcggcggagatacactgtacgccgattctgtgcgcggcaggttcaccaactccagagacaacagcaagaacacgctctatctccagatgaatagcctgcgggccgaagatacagccgtgtactactgtgctgcccggcagggccagtacatctacagcagcatgagaagcgacagctatgactattggggacagggaaccctcgtgaccgtgtcctct

SEQ ID NO: 3 - protein, 123 aa, Artificial Sequence
  Description of Artificial Sequence: Synthetic polypeptide
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 4 - protein, 8 aa, Artificial Sequence
  Description of Artificial Sequence: Synthetic peptide
GRPVSNYA

SEQ ID NO: 5 - protein, 8 aa, Artificial Sequence
  Description of Artificial Sequence: Synthetic peptide
INWQKTAT

SEQ ID NO: 6 - protein, 17 aa, Artificial Sequence
  Description of Artificial Sequence: Synthetic peptide
AAVFRVVAPKTQYDYDY

SEQ ID NO: 7 - protein, 126 aa, Artificial Sequence
  Description of Artificial Sequence: Synthetic polypeptide
EVQLVESGGGLVQPGGSLRLSCAASGRAHSDYAMAWFRQAPGQEREFVAGIGWSGGDTLYADSVRGRFTNSRDNSKNTLYLQMNSLRAEDTAVYYCAARQGQYIYSSMRSDSYDYWGQGTLVTVSS

SEQ ID NO: 8 - protein, 10 aa, Artificial Sequence
  Description of Artificial Sequence: Synthetic peptide
GRAHSDYAMA

SEQ ID NO: 9 - protein, 17 aa, Artificial Sequence
  Description of Artificial Sequence: Synthetic peptide
GIGWSGGDTLYADSVRG

SEQ ID NO: 10 - protein, 19 aa, Artificial Sequence
  Description of Artificial Sequence: Synthetic peptide
AARQGQYIYSSMRSDSYDY

SEQ ID NO: 11 - protein, 15 aa, Artificial Sequence
  Description of Artificial Sequence: Synthetic peptide
GGGGAGGGGAGGGGS

SEQ ID NO: 12 - protein, 6 aa, Artificial Sequence
  Description of Artificial Sequence: Synthetic 6xHis tag
HHHHHH

(19) United States
(12) Patent Application Publication
(10) Pub. No.: US 2023/0357442 A1
(43) Pub. Date: Nov. 9, 2023

(54) METHODS OF TREATING DERMATOMYOSITIS

(71) Applicant: Alexion Pharmaceuticals, Inc., Boston, MA (US)

(72) Inventor: Paul P. Tamburini, Kensington, CT (US)

(73) Assignee: Alexion Pharmaceuticals, Inc., Boston, MA (US)

(21) Appl. No.: 18/029,525

(22) PCT Filed: Oct. 5, 2021
(86) PCT No.: PCT/US2021/053540

§ 371 Date: Mar. 30, 2023

Related U.S. Application Data
(60) Provisional application No. 63/087,577, filed on Oct. 5, 2020.

(51) Int. Cl. A61M 5/28; A61P 17/00; A61M 5/20; C07K 16/46; A61P 21/00

(52) U.S. Cl. CPC C07K 16/468; A61M 5/20; A61M 5/28; A61P 17/00; A61P 21/00; C07K 2317/31; C07K 2317/565

(57) ABSTRACT

The disclosure provides methods of treating a subject having or suspected of having dermatomyositis using a composition including a bi-specific fusion protein. The composition may be administered using a medical device, such as a wearable injector.

### CROSS REFERENCE TO RELATED APPLICATIONS

This application claims the benefit of U.S. Provisional Application No. 63/087,577, filed Oct. 5, 2020, the contents of which is incorporated by reference herein in its entirety.

### BACKGROUND OF THE INVENTION

Dermatomyositis is a rare inflammatory disease most common in adults between the ages of 40 and 60 and in children between the ages of 5 and 15. The major symptom of dermatomyositis is muscle weakness, most often affecting the trunk and muscles closest to the trunk. The affected muscles may be stiff, sore, and/or tender and, eventually, may show signs of degeneration n and muscle wasting (atrophy). Muscle weakness and degeneration typically progress and may lead to a gradual inability to perform certain tasks, such as lifting the arms, climbing steps, or dressing. Individuals with dermatomyositis also develop characteristic skin changes that, in some cases, may precede muscle weakness. These characteristic skin changes may be the only sign of dermatomyositis at disease onset in up to 40% of people. Skin abnormalities include a distinctive skin rash on the face, eyelids, chest, back, knuckles, knees, or elbows that is typically patchy and reddish-purple in color and that can be itchy or painful. Other symptoms of dermatomyositis include muscle pain, muscle tenderness, difficulty swallowing, breathing problems, hard calcium deposits underneath the skin, fatigue, unintentional weight loss, and fever.

There is no cure for dermatomyositis and treatment is focused on improving symptoms. Corticosteroids, immunosuppressive drugs, and anti-malarial drugs have been used to treat subjects diagnosed with dermatomyositis; however, these agents can have serious adverse effects, particularly when administered at high doses and/or for extended periods of time. Accordingly, there is a need for new therapies to treat dermatomyositis.

### SUMMARY OF THE INVENTION

The invention provides methods for treating a subject (e.g., a human subject) having or suspected of having dermatomyositis by administering a composition containing a bi-specific fusion protein that binds to complement component C5 (C5) and human serum albumin (HSA). The bi-specific fusion protein can be administered using a medical device; accordingly, the invention also features medical devices containing a composition containing the bi-specific fusion protein. The medical device can be used to administer the composition to the subject by any suitable route, such as by subcutaneous injection.

In a first aspect, the invention provides a method of treating a subject having or suspected of having dermatomyositis by administering to the subject a pharmaceutical composition containing a therapeutically effective amount of a fusion protein having the sequence of SEQ ID NO:1 and a pharmaceutically acceptable carrier. In another aspect, the invention provides a medical device for treating a subject having or suspected of having dermatomyositis, the medical device containing a pharmaceutical composition containing the fusion protein of SEQ ID NO:1 (e.g., a therapeutically effective amount of the fusion protein) and a pharmaceutically acceptable carrier. In some embodiments, the device is an autoinjector or a pre-filled syringe. In some embodiments, the pre-filled syringe is a multi-chambered pre-filled syringe or a lyo syringe (i.e., one containing the lyophilized form of the fusion protein described herein). In some embodiments, the device is a wearable device (e.g., a wearable injector).

In another aspect, the invention provides a therapeutic kit containing: (a) a container including a label; and (b) a composition containing the fusion protein of SEQ ID NO:1 (e.g., a therapeutically effective amount of the fusion protein) and a pharmaceutically acceptable carrier; in which the label indicates that the composition is to be administered to a subject having or suspected of having dermatomyositis.

In some embodiments of any of the foregoing aspects, the dermatomyositis is juvenile dermatomyositis. In some embodiments of any of the foregoing aspects, the dermatomyositis is adult dermatomyositis. In some embodiments of any of the foregoing aspects, the dermatomyositis is amyopathic dermatomyositis. In some embodiments of any of the foregoing aspects, the dermatomyositis is associated with cancer (malignancy-associated dermatomyositis).

The fusion proteins described herein provide several advantages. For example, therapeutic use of the anti-HSA VHH-anti-C5 VHH fusion protein described herein provides a number of advantages. The anti-HSA VHH domain provides the fusion protein with improved stability and a longer half-life to as compared to therapeutics that do not include an HSA binding moiety. In addition, because the fusion protein is made up of two VHH domains, it is much smaller than a conventional four-chain antibody, which includes both VH and VL domains (VHH domains are approximately 10 times smaller than a conventional IgG). The small size allows for higher penetration into tissues than conventional four-chain antibodies and antigen-binding fragments thereof, and VHH domains are also known to be able to access targets and epitopes not accessible to conventional four-chain antibodies and antigen-binding fragments thereof. In addition, VHH domains are highly soluble and do not have a tendency to aggregate. They are also highly stable to heat, pH, proteases, and other denaturing agents or conditions. Consequently, the fusion protein described herein can be administered at a higher dose than a conventional four-chain antibody or an antigen-binding fragment thereof. Finally, the anti-HSA VHH-anti-C5 VHH fusion protein targets a specific protein in the complement pathway. Accordingly, it may be used to treat subjects having dermatomyositis at a higher dose, for a longer time, and/or with fewer adverse effects than the anti-inflammatory or immunosuppressive agents currently used to treat dermatomyositis.

Other features and advantages of the invention will be apparent from the following Detailed Description and the Claims.

### DEFINITIONS

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “binding domain” refers to the portion of a protein or antibody which comprises the amino acid residues that interact with an antigen. Binding domains include, but are not limited to, antibodies (e.g., full length antibodies), as well as antigen-binding portions thereof. The binding domain confers on the binding agent its specificity and affinity for the antigen. The term also covers any protein having a binding domain which is homologous or largely homologous to an immunoglobulin-binding domain.

The term “antibody” as used herein includes whole antibodies and any antigen binding fragment (i.e., “antigen-binding portion”) or single chain version thereof. An “antibody” refers, in one preferred embodiment, to a glycoprotein comprising at least two heavy (H) chains and two light (L) chains inter-connected by disulfide bonds, or an antigen binding portion thereof. The terms “heavy chain” and “light chain,” as used herein, refer to any Ig polypeptide having sufficient variable domain sequence to confer specificity for a target antigen. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody (or simply “antibody fragment”), as used herein, refers to one or more fragments or portions of an antibody that retain the ability to specifically bind to an antigen. Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An antibody, immunoglobulin, or immunologically functional immunoglobulin fragment, or the engineered polypeptides or fusion proteins disclosed herein, are said to “specifically” bind an antigen when the molecule preferentially recognizes its antigen target in a complex mixture of proteins and/or macromolecules. The term “specifically binds,” as used herein, refers to the ability of an antibody, immunoglobulin, or immunologically functional immunoglobulin fragment, or an engineered polypeptide or fusion protein of the disclosure, to bind to an antigen containing an epitope with an K_{D} of at least about 10⁻⁶ M,10⁻⁷ M, 10⁸ M, 10⁻⁹ M, 10⁻¹⁰ M, 10⁻¹¹ M, 10⁻¹² M, or more, and/or to bind to an epitope with an affinity that is at least two-fold greater than its affinity for a nonspecific antigen.

The term “heavy chain antibody,” as used herein, refers to an antibody that consists of two heavy chains and lacks the two light chains found in conventional antibodies.

The term “VHH domain” refers to variable domains present in naturally occurring heavy chain antibodies to distinguish them from the heavy chain variable domains that are present in conventional four chain antibodies (referred to herein as “VH domains”) and from the light chain variable domains that present in conventional four chain antibodies (referred to herein as “VL domains”). Single domain, heavy chain variable domain sequences from a heavy chain antibody may be referred to as VHH or V_{H}H antibodies, VHH or V_{H}H antibody fragments, or VHH or V_{H}H domains.

The term “peptide linker,” as used herein, refers to one or more amino acid residues inserted or included between the polypeptides of a fusion protein. The peptide linker can be, for example, inserted or included at the transition between the polypeptides of the fusion protein at the sequence level. The identity and sequence of amino acid residues in the linker may vary depending on the desired secondary structure. For example, glycine, serine and alanine are useful for linkers having maximum flexibility. Any amino acid residue can be considered as a linker in combination with one or more other amino acid residues, which may be the same as or different from the first amino acid residue, to construct larger peptide linkers as necessary depending on the desired properties.

The term “half-life,” as used herein, refers to the time taken for the serum concentration of the fusion protein to be reduced by 50%, in vivo, as a result, for example, of the degradation of the molecule and/or clearance or sequestration of the molecule by physiological mechanisms. Methods for pharmacokinetic analysis and determination of half-life are known to those skilled in the art.

The term “bispecific” refers to a fusion protein that is capable of binding two antigens. The term “multivalent fusion protein” means a fusion protein comprising two or more antigen binding sites. The term “multi-specific fusion protein” refers to a fusion protein that is capable of binding two or more related or unrelated targets.

The term “vector,” as used herein, refers to any molecule (e.g., nucleic acid, plasmid or virus) that is used to transfer coding information to an expression system (e.g., a host cell or in vitro expression system). One type of vector is a “plasmid,” which refers to a circular double-stranded DNA (dsDNA) molecule into which additional DNA segments can be inserted. Another type of vector is a viral vector, wherein additional DNA segments can be inserted into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of coding sequences to which they are operatively linked. Such vectors are referred to herein as “expression vectors.”

The term “operably linked,” as used herein, refers to an arrangement of flanking sequences wherein the flanking sequences are configured or assembled to perform a desired function. Thus, a flanking sequence operably linked to a coding sequence may be capable of effecting the replication, transcription, and/or translation of the coding sequence. A coding sequence is operably linked to a promoter, for example, where the promoter is capable of directing transcription of that coding sequence. A flanking sequence need not be contiguous with the coding sequence to be considered operably linked, so long as it functions correctly.

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not be, in fact, identical to the parent cell, but such cells are still included within the scope of the term “host cell” as used herein. A wide variety of host cell expression systems can be used to express the fusion protein of the disclosure, including bacterial, yeast, baculoviral, and mammalian expression systems (as well as phage display expression systems).

The terms “patient” and “subject” as used herein include human and animal subjects.

The terms “pharmaceutical composition” or “therapeutic composition,” as used herein, refer to a compound or composition capable of inducing a desired therapeutic effect when administered to a patient.

The term “pharmaceutically acceptable carrier” or “physiologically acceptable carrier,” as used herein, refers to one or more formulation materials suitable for accomplishing or enhancing the delivery of the fusion protein of the disclosure.

The terms “treatment” or “treat,” as used herein, refer to both therapeutic treatment and prophylactic or preventative measures. Those in need of treatment include those having a disease or condition as well as those at risk of having the disease or condition or those in which the disease or condition is to be prevented.

As used herein, a “therapeutically effective” amount of, for example, a fusion protein described herein, refers to an amount or dosage sufficient to produce a desired therapeutic result. A therapeutically effective amount is an amount is an amount that, when administered, results in a decrease in severity of disease symptoms (e.g., a decrease in symptoms of dermatomyositis, an increase in frequency and duration of disease symptom free periods, or a prevention of impairment or disability due to the disease affliction) or that is sufficient to inhibit, for some period of time, one or more of the clinically defined pathological processes associated with the condition being treated, e.g., dermatomyositis. In certain embodiments, a therapeutically effective amount of a therapeutic agent described herein can include an amount (or various amounts in the case of multiple administrations) that reduces a dermatomyositis-associated rash or that reduces muscle weakness and/or atrophy. The therapeutically effective amount may vary depending on a variety of factors and conditions related to the patient being treated and the severity of the disorder.

### DETAILED DESCRIPTION OF THE INVENTION

The invention features methods of treating dermatomyositis using a fusion protein including an anti-human serum albumin (HSA) single domain, heavy chain variable domain sequence from a heavy chain antibody (referred to herein as a VHH domain) and an anti-complement component C5 (C5) VHH domain. The two VHH domains are joined by a peptide linker. The fusion protein can be used to treat any form of dermatomyositis, such as adult dermatomyositis, juvenile myositis, or amyopathic dermatomyositis (also known as dermatomyositis sine myositis). Because the fusion protein specifically binds to HSA and C5, it can have a more targeted effect on the immune system than the anti-inflammatory and immunosuppressive agents currently used to treat dermatomyositis. Accordingly, the fusion protein of the invention may be used to treat subjects having dermatomyositis at a higher dose, for a longer time, and/or with fewer adverse effects than the agents currently used to treat dermatomyositis.

### Dermatomyositis

Dermatomyositis is a rare inflammatory disease marked by muscle weakness and a distinctive skin rash. The disease is most common in adults between the ages of 40 and 60 and in children between the ages of 5 and 15 and it is known to affect women more than men. Muscle weakness most often affects the trunk and muscles closest to the trunk (i.e., proximal muscles), such as the hips, thighs, shoulders, upper arms, and neck. The affected muscles may be stiff, sore, and/or tender and, eventually, may show signs of degeneration and muscle wasting (atrophy). Muscle weakness and degeneration typically progress and may lead to changes in gait and a gradual inability to perform certain tasks, such as lifting the arms, climbing steps, or dressing. Individuals with dermatomyositis also develop characteristic skin changes that, in some cases, may precede muscle weakness. These characteristic skin changes may be the only sign of dermatomyositis at disease onset in up to 40% of people. Skin abnormalities include a distinctive skin rash on the face, eyelids, chest, back, knuckles, knees, or elbows that is typically patchy and reddish-purple in color and that can be itchy or painful. This rash may be referred to as a heliotrope rash. Other symptoms of dermatomyositis include muscle pain, muscle tenderness, difficulty swallowing (dysphagia), difficulty articulating speech (dysphonia), breathing problems, hard calcium deposits underneath the skin, joint pain, fatigue, unintentional weight loss, and fever.

There is no cure for dermatomyositis and treatment is focused on improving symptoms. Medications used to treat dermatomyositis include corticosteroids (e.g., prednisone), immunosuppressive agents (e.g., azathioprine, methotrexate, mycophenolate mofetil, cyclophosphamide, tacrolimus, or cyclosporine), rituximab, and anti-malarial drugs (e.g., hydroxychloroquine). Such medications may be used together with the methods of administering the fusion proteins described herein. Surgery and intravenous infusion of immunoglobulin (IVIg) have also been used to treat dermatomyositis. Such procedures also may be used together with the methods of administering the fusion proteins described herein. The specific underlying cause(s) of dermatomyositis remain unknown; however, dermatomyositis is thought to be an autoimmune disorder and evidence suggests the involvement of genetic, environmental, and immune factors. For example, microvascular complement deposition has been observed in subjects with dermatomyositis, but how the complement system is activated, the predominant complement pathway involved, and the role of complement deposition in disease pathogenesis have yet to be determined.

### Anti-C5 Bi-Specific Fusion Protein

Therapeutics that target the complement system provide a new approach for dermatomyositis treatment. The complement system acts in conjunction with other immunological systems of the body to defend against intrusion of cellular and viral pathogens. There are at least 25 complement proteins, which are a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade can progress via the classical pathway (CP), the lectin pathway or the alternative pathway (AP). The lectin pathway is typically initiated with binding of mannose-binding lectin (MBL) to high mannose substrates. The AP can be antibody independent and initiated by certain molecules on pathogen surfaces. The CP is typically initiated by antibody recognition of, and binding to, an antigenic site on a target cell. These pathways converge at the C3 convertase- where complement component C3 is cleaved by an active protease to yield C3a and C3b.

Described herein is a bi-specific fusion protein that binds to a complement protein that is cleaved by a complex containing C3b, complement component C5 (C5). C5 is composed of alpha and beta polypeptide chains and is cleaved during activation of both the classical and alternative complement pathways. In addition to binding to C5, the bi-specific fusion protein described herein also binds to human serum albumin (HSA) to increase the half-life of the protein. The anti-C5 and anti-HSA binding moieties in the fusion protein are VHH domains, each including three CDR sequences. The anti-C5 and anti-HSA VHH domains are joined by a peptide linker to form the fusion protein.

The anti-HSA VHH-anti-C5 VHH fusion protein has the sequence:

EVQLVESGGGLVKPGGSLRLSCAASGRPVSNYAAAWFRQAPGKEREFVSA
INWQKTATYADSVKGRFTISRDNAKNSLYLQMNSLRAEDTAVYYCAAVFR
VVAPKTQYDYDYWGQGTLVTVSSGGGGAGGGGAGGGGSEVQLVESGGGLV
QPGGSLRLSCAASGRAHSDYAMAWFRQAPGQEREFVAGIGWSGGDTLYAD
SVRGRFTNSRDNSKNTLYLQMNSLRAEDTAVYYCAARQGQYIYSSMRSDS
YDYWGQGTLVTVSS (SEQ ID NO:1).

It is encoded by a polynucleotide having the sequence:

GAGGTGCAGCTGGTTGAATCTGGCGGAGGACTTGTGAAGCCTGGCGGCTC
TCTGAGACTGTCTTGTGCTGCTTCTGGCAGACCCGTGTCTAATTACGCCG
CTGCCTGGTTTAGACAGGCCCCTGGCAAAGAGAGAGAGTTCGTCAGCGCC
ATCAACTGGCAGAAAACCGCCACATACGCCGACAGCGTGAAGGGCAGATT
CACCATCAGCCGGGACAACGCCAAGAACAGCCTGTACCTGCAGATGAACT
CCCTGAGAGCCGAGGACACCGCCGTGTATTATTGTGCCGCCGTGTTTAGA
GTGGTGGCCCCTAAGACACAGTACGACTACGATTACTGGGGCCAGGGCAC
CCTGGTTACAGTTTCTAGTGGCGGAGGCGGAGCTGGTGGTGGCGGAGCAG
GAGGCGGAGGATCTGAAGTTCAACTTGTCGAGAGTGGCGGCGGACTGGTT
CAACCAGGCGGAAGTCTTAGACTGAGCTGTGCTGCTAGCGGCAGAGCCCA
CTCTGATTACGCTATGGCATGGTTCAGGCAGGCCCCAGGACAAGAAAGGG
AATTTGTGGCCGGAATCGGTTGGAGCGGCGGAGATACACTGTACGCCGAT
TCTGTGCGCGGCAGGTTCACCAACTCCAGAGACAACAGCAAGAACACGCT
CTATCTCCAGATGAATAGCCTGCGGGCCGAAGATACAGCCGTGTACTACT
GTGCTGCCCGGCAGGGCCAGTACATCTACAGCAGCATGAGAAGCGACAGC
TATGACTATTGGGGACAGGGAACCCTCGTGACCGTGTCCTCT (SEQ ID
NO: 2).

The portion of the fusion protein that corresponds to the anti-HSA binding domain has the sequence:

EVQLVESGGGLVKPGGSLRLSCAASGRPVSNYAAAWFRQAPGKEREFVSAINWQKTATYA DSVKGRFTISRDNAKNSLYLQMNSLRAEDTAVYYCAAVFRVVAPKTQYDYDYWGQGTLVTVSS (SEQ ID NO: 3). The anti-HSA binding domain contains three CDR sequences, a first CDR (CDR1) having the sequence: GRPVSNYA (SEQ ID NO: 4), a second CDR (CDR2) having the sequence: INWQKTAT (SEQ ID NO: 5), and a third CDR (CDR3) having the sequence: AAVFRVVAPKTQYDYDY (SEQ ID NO: 6).

The portion of the fusion protein that corresponds to the anti-C5 binding domain has the sequence:

EVQLVESGGGLVQPGGSLRLSCAASGRAHSDYAMAWFRQAPGQEREFVAGIGWSGGDTL YADSVRGRFTNSRDNSKNTLYLQMNSLRAEDTAVYYCAARQGQYIYSSMRSDSYDYWGQGTLVTV SS (SEQ ID NO: 7). The anti-C5 binding domain also contains three CDR sequences, a first CDR (CDR1) having the sequence: GRAHSDYAMA (SEQ ID NO: 8), a second CDR (CDR2) having the sequence: GIGWSGGDTLYADSVRG (SEQ ID NO: 9), and a third CDR (CDR3) having the sequence: AARQGQYIYSSMRSDSYDY (SEQ ID NO: 10).

The anti-HSA binding domain and the anti-C5 binding domain are joined by a peptide linker having the sequence: GGGGAGGGGAGGGGS (SEQ ID NO: 11).

### Vectors, Host Cells, and Protein Production

The fusion protein of the invention can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Host cells can be mammalian, plant or microbial in origin. In addition to known mammalian host cells, yeast host cells, e.g., Pichia pastoris, Saccharomyces cerevisiae, and/or plant host cells can be used. Generally, preferred host cells are of either eukaryotic (e.g., mammalian or yeast) or prokaryotic (e.g., bacterial) origin.

A general description of multivalent and multi-specific fusion proteins containing one or more VHH antibodies and their preparation are known (Els Conrath, K. et al., J. Biol. Chem., 276:7346-50, 2001; Muyldermans, S., J. Biotechnol., 74:277-302 2001; International Publication Nos. WO 96/34103, WO 99/23221 and WO 04/041865). The fusion protein described herein can be expressed from or associated with constructs that include, for example, one or more elements such as expression vectors (WO 04/041862).

### Nucleic Acid Vector Construction and Host Cells

A nucleic acid sequence encoding the fusion protein described herein may be prepared by a variety of methods known in the art. These methods include, but are not limited to, oligonucleotide-mediated (or site-directed) mutagenesis, PCR mutagenesis, ligation, and overlap extension PCR. A nucleic acid molecule encoding the fusion protein described herein may be obtained using standard techniques, e.g., gene synthesis. Nucleic acid molecules can be synthesized using a nucleotide synthesizer or PCR techniques.

A nucleic acid sequence encoding the fusion protein may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In some embodiments, yeast cells may be used as host cells for the invention. Exemplary yeast host cells include Pichia pastoris and Saccharomyces cerevisiae. In some embodiments, E. coli cells may also be used as host cells for the invention. Examples of E. coli strains include, but are not limited to, E. coli 294 (ATCC^{®} 31,446), E. coli λ 1776 (ATCC^{®} 31,537, E. coli BL21 (DE3) (ATCC^{®} BAA-1025), and E. coli RV308 (ATCC^{®} 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the fusion protein of the invention may be grown in media known in the art and suitable for culturing of the selected host cells. Examples of suitable media for mammalian host cells include Minimal Essential Medium (MEM), Dulbecco’s Modified Eagle’s Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. The fusion protein may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the fusion protein described herein can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the fusion protein of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions

The fusion protein described herein (e.g., a fusion protein having the sequence of SEQ ID NO: 1) can be incorporated into a vehicle for administration into a patient, such as a human patient suffering from dermatomyositis. Pharmaceutical compositions containing the fusion protein can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures containing the fusion protein described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in US 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Acceptable formulation materials can be used to modify, maintain, or preserve, for example, the pH, osmolarity, viscosity, clarity, color, isotonicity, odor, sterility, stability, rate of dissolution or release, adsorption, or penetration of the composition. Acceptable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCI, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides — preferably sodium or potassium chloride — or mannitol sorbitol), delivery vehicles, diluents, excipients and/or pharmaceutical adjuvants (see, e.g., Remington: The Science and Practice of Pharmacology 22nd edition, Allen, L. Ed. (2013); incorporated herein by reference.

A skilled artisan can develop a pharmaceutical composition containing the fusion protein described herein depending upon, for example, the intended route of administration, delivery format, and desired dosage. Pharmaceutical compositions described herein can include one or more agents to improve, for example, delivery of the therapeutic agent. Agents that degrade hyaluronan, for example, can be included in the pharmaceutical compositions described herein, or such agents can be co-administered with the pharmaceutical compositions described herein to facilitate, for example, dispersion and absorption of the therapeutic agents described herein upon administration. An example of such an agent is recombinant hyaluronidase.

Additional pharmaceutical compositions will be evident to those of skill in the art, including formulations involving sustained-delivery or controlled-delivery formulations. Techniques for formulating sustained-delivery or controlled-delivery formulations, using, for example, liposome carriers, bio-erodible microparticles or porous beads, and depot injections, are known to those of skill in the art.

A solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein can be administered to a subject having or suspected of having dermatomyositis by a variety of routes, such as intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The composition can also be administered topically (e.g., to an area of the skin exhibiting a rash). In some embodiments, the composition is administered subcutaneously (e.g., via subcutaneous injection or infusion). The most suitable route for administration in any given case will depend on the particular composition administered, the patient, pharmaceutical formulation methods, administration methods (e.g., administration time and administration route), the patient’s age, body weight, sex, severity of the disease being treated, the patient’s diet, and the patient’s excretion rate. Compositions may be administered once, or more than once (e.g., once annually, twice annually, three times annually, bimonthly, monthly, bi-weekly, or weekly).

Subjects that may be treated as described herein are subjects having dermatomyositis (e.g., subjects identified as having dermatomyositis) and subjects suspected of having dermatomyositis, including adult dermatomyositis, juvenile dermatomyositis, and amyopathic dermatomyositis (also known as dermatomyositis sine myositis). In some embodiments, the dermatomyositis is associated with cancer (malignancy-associated dermatomyositis), which may precede, occur in association with, or develop subsequent to the onset of dermatomyositis. If a subject is suspected of having dermatomyositis, one or more of the following tests can be performed to aid in diagnosis: a blood test to measure muscle enzymes associated with muscle damage (e.g., creatine kinase) or to detect autoantibodies, a chest X-ray, electromyography, magnetic resonance imaging (MRI), a skin biopsy, or a muscle biopsy. Treatment of dermatomyositis can lead to an improvement in dermatomyositis symptoms. For example, treatment with the fusion protein described herein may treat the skin abnormalities (e.g., rash) associated with dermatomyositis and/or the muscle abnormalities (e.g., muscle weakness and/or atrophy) associated with dermatomyositis. In some embodiments, the compositions and methods described herein can reduce or eliminate skin abnormalities, e.g., reduce the extent of a rash associated with dermatomyositis (reduce the amount of skin exhibiting a rash), reduce pain associated with the rash, reduce itching associated with the rash, and/or reduce scaly, dry, or rough skin associated with dermatomyositis. In some embodiments, the compositions and methods described herein reduce muscle weakness and/or atrophy (e.g., improve muscle strength). The compositions and methods described herein can also be used to slow or inhibit disease progression (e.g., to slow or inhibit the weakening and/or atrophy of muscles). In some embodiments, the compositions and methods described herein prevent or reduce other symptoms of dermatomyositis, such as muscle pain, muscle tenderness, muscle stiffness, muscle inflammation, the development of contractures, inflamed or swollen areas around fingernails, difficulty swallowing (dysphagia), difficulty articulating speech (dysphonia), breathing problems, the formation of hard calcium deposits in muscles, skin, or connective tissue, joint pain, fatigue, unintentional weight loss, or fever. The compositions and methods described herein can also prevent the development of, reduce the likelihood of developing, or reduce the severity of conditions associated with dermatomyositis, such as Raynaud’s phenomenon, other connective tissue diseases, cardiovascular disease (e.g., heart muscle inflammation), lung disease (e.g., interstitial lung disease), or cancer (e.g., ovarian cancer, lung cancer, testicular cancer, or cancer of the GI tract). Skin abnormalities (e.g., a rash) can be assessed using visual inspection or a skin biopsy and muscle abnormalities (e.g., muscle weakness and/or atrophy) can be assessed by monitoring the performance of tasks that may be impaired in subjects suffering from dermatomyositis (e.g., gait, lifting the arms, climbing steps, dressing, raising the head from a pillow, rising unassisted from the floor), by measuring the level of muscle enzymes in the blood that can indicate muscle damage (e.g., creatine kinase, aldolase, aspartate aminotransferase, or lactic dehydrogenase), or by performing electromyography or a muscle biopsy. Skin abnormalities and/or muscle abnormalities can be assessed before and after treatment with the compositions described herein to monitor response to treatment.

Treatment may include administration of a composition containing the fusion protein described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may comprise continuous infusion over a set period of time. The composition can include a dosage of the fusion protein of the invention ranging from 0.5 mg/kg to 50 mg/kg (e.g., 1.0, 2.0, 3.0, 4.0, 5.0, 6.0, 7.0, 8.0, 9.0 10.0, 12.0, 15.0, 18.0, 20.0, 25.0, 30.0, 35.0, 40.0, 45.0, or 50.0 mg/kg). In some embodiments, the amount of the fusion protein administered to the subject is about 10 mg to about 3,000 mg (e.g., about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, or 3,000 mg). The dosage may be adapted by the physician in accordance with conventional factors such as the extent of the disease and different parameters of the subject.

The compositions described herein are administered in an amount sufficient to improve one or more symptom of dermatomyositis (e.g., skin abnormalities, such as a rash or scaly, dry, or rough skin, muscle abnormalities, such as muscle weakness, muscle atrophy, muscle pain, muscle tenderness, muscle stiffness, or muscle inflammation, inflamed or swollen areas around fingernails, the development of contractures, difficulty swallowing (dysphagia), difficulty articulating speech (dysphonia), breathing problems, the formation of hard calcium deposits in muscles, skin, or connective tissue, joint pain, fatigue, unintentional weight loss, or fever) or to slow or inhibit disease progression (e.g., progressive muscle weakening or atrophy). These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. Depending on the outcome of the evaluation, the patient may receive additional treatments.

The compositions described herein can be administered by a medical professional or self-administered. For self-administration, the composition can be included in a medical device, such as an autoinjector or a pre-filled syringe. In some embodiments, the medical device is a wearable device, such as a wearable injector. Exemplary wearable injectors include the SmartDose® injectors developed by West Pharmaceutical Services, which can be used for pre-programmed subcutaneous injection, and the BD Libertas™ wearable injector. In some embodiments, the medical device is a wearable infusion device, such as an infusion device produced by Enable Injections to allow subcutaneous, self-administration of high volume therapeutics. The composition can be contained in a cartridge for loading into an autoinjector or a wearable injector.

The fusion protein described herein can be administered in combination with an additional therapy. In some embodiments, the therapy is a medication currently used to treat dermatomyositis, such as a corticosteroid (e.g., prednisone), an immunosuppressive agent (e.g., azathioprine, methotrexate, mycophenolate mofetil, cyclophosphamide, tacrolimus, or cyclosporine), rituximab, or an anti-malarial drug (e.g., hydroxychloroquine). The fusion protein may also be administered in combination with intravenous immunoglobulin (IVIg) or administered to a subject in combination with surgery to remove calcium deposits. In some embodiments, the therapy is physical therapy, a dietary modification, speech therapy, or avoidance of and/or protection from the sun (e.g., use of sunscreen). Coadministration of the fusion protein described herein and an addition therapy may be simultaneous (e.g., administered concurrently or in a single pharmaceutical composition), separate, or sequential (e.g., the fusion protein described herein may be administered before or after the additional therapy).

### Kits

A composition containing the fusion protein described herein can be provided in a kit for use in treating dermatomyositis. The kit can further include a label or package insert that instructs a user of the kit, such as a subject having dermatomyositis or a physician, to perform the methods described herein. In some embodiments, the kit includes a container having a label and a composition including the fusion protein described herein and the label indicates that the composition is to be administered to a patient having, or that is suspected of having, dermatomyositis. The kit may optionally include a syringe or other device (e.g., autoinjector, pre-filled syringe, or wearable device) for administering the composition. In some embodiments, the kit includes both a first container having a dried protein and a second container having an aqueous formulation. In other embodiments, the kit includes single or multi-chambered pre-filled syringes (e.g., liquid syringes and lyosyringes). In some embodiments, the kit includes cartridges containing a composition of the invention for use with a medical device (e.g., an autoinjector or a wearable device).

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Administration of a Composition Containing an Anti-HSA VHH-Anti-C5 VHH Fusion Protein

According to the methods disclosed herein, a physician of skill in the art can treat a patient, such as a human patient, with dermatomyositis so as to reduce one or more symptom of dermatomyositis (e.g., reduce a rash associated with dermatomyositis or reduce muscle weakness and/or atrophy) or slow disease progression. To this end, a physician of skill in the art can administer to the human patient a composition containing a fusion protein having the sequence of SEQ ID NO: 1. The composition containing the fusion protein may be administered to the patient, for example, by injection, such as intravenous or subcutaneous injection. The composition can be provided in a medical device, such as an autoinjector or a wearable medical device, so the patient may self-administer the composition. The fusion protein is administered in a therapeutically effective amount, such as from 0.5 mg/kg to 50 mg/kg (e.g., 1.0, 2.0, 3.0, 4.0, 5.0, 6.0, 7.0, 8.0, 9.0 10.0, 12.0, 15.0, 18.0, 20.0, 25.0, 30.0, 35.0, 40.0, 45.0, or 50.0 mg/kg) or 10 mg to 3,000 mg (e.g., about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, or 3,000 mg. The fusion protein is administered in an amount sufficient to reduce one or more symptom of dermatomyositis and/or slow progression of the disease.

Following administration of the composition to a patient, a practitioner of skill in the art can monitor the patient’s improvement in response to the therapy, by a variety of methods. For example, a physician can monitor the patient’s a rash using visual inspection or a skin biopsy and the patient’s muscle weakness by monitoring the patient’s performance of tasks that are typically impaired in dermatomyositis (e.g., gait, lifting the arms, climbing steps, dressing, raising the head from a pillow, rising unassisted from the floor), by measuring the level of muscle enzymes in the blood that can indicate muscle damage (e.g., creatine kinase), or by performing electromyography or a muscle biopsy. A finding that the patient exhibits a reduction in the rash, muscle weakness, and/or muscle atrophy following administration of the composition or that the disease has not progressed indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of treating a subject having or suspected of having dermatomyositis, comprising administering to the subject a pharmaceutical composition comprising a therapeutically effective amount of a fusion protein having the sequence of SEQ ID NO:1 and a pharmaceutically acceptable carrier.

2. A medical device for treating a subject having or suspected of having dermatomyositis, the medical device comprising a pharmaceutical composition comprising the fusion protein of SEQ ID NO:1 and a pharmaceutically acceptable carrier.

3. The medical device of claim 2, wherein the device is an autoinjector or a pre-filled syringe.

4. The medical device of claim 3, wherein the pre-filled syringe is a multi-chambered pre-filled syringe or a lyo syringe.

5. The medical device of claim 2, wherein the device is a wearable device.

6. A therapeutic kit comprising:
(a) a container comprising a label; and
(b) a composition comprising the fusion protein of SEQ ID NO:1 and a pharmaceutically acceptable carrier;
wherein the label indicates that the composition is to be administered to a subject having, or who is suspected of having dermatomyositis.
